(19) United States
(12) Patent Application Publication
(10) Pub. No.: US 2022/0047552 A1
(43) Pub. Date: Feb. 17, 2022

(54) RAF-1 KINASE INHIBITOR COMPOUNDS FOR SKELETAL MUSCLE MODULATION, METHODS AND USES THEREOF

(71) Applicant: SOCIETE DES PRODUITS NESTLE S.A., Vevey (CH)

(72) Inventors: Denis Marcel Barron, Lutry (CH); Jerome Feige, Crissier (CH); Sonia Karaz, Epalinges (CH); Joris Michaud, Lausanne (CH); Yann Ratinaud, Morges (CH); Pascal Stuelsatz, Crissier (CH)

(21) Appl. No.: 17/276,557

(22) PCT Filed: Sep. 12, 2019
(86) PCT No.: PCT/EP2019/074328

§ 371 Date: Mar. 16, 2021

(30) Foreign Application Priority Data
Sep. 18, 2018 (EP) 18195273.0

(51) Int. Cl. A61K 31/4045; A61P 21/06

(52) U.S. Cl. CPC A61K 31/4045; A61P 21/06

(57) ABSTRACT

The present invention relates to novel Raf-1 kinase inhibitor compounds for improving skeletal muscle regeneration to maintain or increase muscle function and/or muscle mass by modulating muscle stem cells. For example, the present invention is useful for subjects to promote muscle repair and/or subjects suffering from precachexia, cachexia, sarcopenia, myopathy, dystrophy and/or recovery after muscle injury or surgery.

### FIELD OF THE INVENTION

The present invention relates to novel Raf-1 kinase inhibitor compounds for improving skeletal muscle regeneration to maintain or increase muscle function and/or muscle mass by modulating muscle stem cells. For example, the present invention is useful for subjects to promote muscle repair and/or subjects suffering from precachexia, cachexia, sarcopenia, myopathy, dystrophy and/or recovery after muscle injury or surgery.

### BACKGROUND TO THE INVENTION

Skeletal muscle regeneration is a crucial mechanism to repair and maintain muscle mass and function throughout life. Skeletal muscle regeneration primarily requires the participation of myogenic progenitors, known as muscle stem cells or satellite cells.

Non-proliferative, quiescent satellite cells, which adjoin resting skeletal muscles, can be identified by their distinct location between sarcolemma and basal lamina, a high nuclear-to-cytoplasmic volume ratio, few organelles (e.g. ribosomes, endoplasmic reticulum, mitochondria, golgi complexes), small nuclear size, and a large quantity of nuclear heterochromatin relative to myonuclei. On the other hand, activated satellite cells have an increased number of caveolae, cytoplasmic organelles, and decreased levels of heterochromatin.

These muscle satellite cells are part of the adult stem cell niche and they are involved in the normal growth of muscle, as well as regeneration following injury or disease. Hence, they are a potential target to enhance muscle regeneration in both healthy and diseased conditions. Skeletal muscle regeneration follows a series of steps that recapitulates the phases of development. Muscle progenitor cells must exit the state of quiescence and become active, proliferate and commit to myogenic differentiation.

Satellite cells express genetic markers at different stages of myogenesis and proliferation. Pax7 and Pax3 are considered to be satellite cell markers. For example, activated satellite cells expressing low levels of Pax7 are more committed to differentiation, whereas high levels of Pax7 are related to cells less prone to differentiate and have more undifferentiated stemness characteristics. Activation and the induction of myogenesis is typically regulated by myogenic regulatory factors such as MyoD, Myf5, myogenin and MRF4. Negative regulation by myostatin and TGFb inhibits the differentiation of satellite cells (Almeida et al., 2016).

Experimental therapies which have previously included myoblast transplantation have not been entirely successful due to the reduced regenerative potential of myoblasts and myofibers which are more committed and differentiated in comparison the muscle stem cells.

Therefore, there remains a significant need to identify compounds, compositions and methods which modulate muscle stem cells directly for maintaining muscle health and improving muscle regeneration. Such compounds, compositions and methods of treatment may help subjects with muscle stem cell dysfunction and/or subjects suffering from muscular diseases and conditions, such as cachexia or sarcopenia by facilitating maintenance of, increasing muscle function and/or muscle mass.

### SUMMARY OF THE INVENTION

The present invention has identified novel compounds and compositions to modulate skeletal muscle function and improve skeletal muscle regeneration in order improve muscle repair after injury or to counteract muscle wasting that occurs in a number of pathological conditions, in particular, cachexia and sarcopenia.

In one embodiment, the present invention relates to a compound of formula (I):

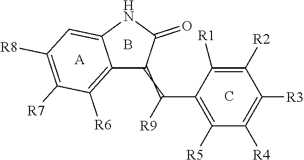

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl.

The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group. The phenyl ring C can be replaced by a pyridyl, pyrimidyl, naphthyl, quinolinyl, or isoquinolinyl group. These groups can be further substituted by H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In another embodiment, the invention relates to compounds of the formula (II):

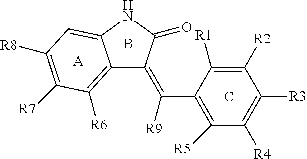

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

The phenyl ring C can be replaced by a pyridyl, pyrimidyl, naphthyl, quinolinyl, or isoquinolinyl group. These groups can be further substituted by H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In another embodiment, the invention relates to compounds of the formula (III):

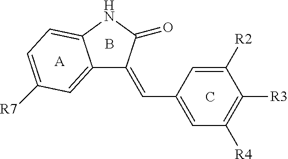

wherein R2, R3, R4, and R7 are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In a preferred embodiment, the invention relates to compounds of formula (III) wherein R2, R4 and R7 are substituted halogens.

In a preferred embodiment of the invention, the compound is 3-(3,5-Dibromo-4-hydroxy-benzylidene)-5-iodo-1,3-dihydro-indo1-2-one:

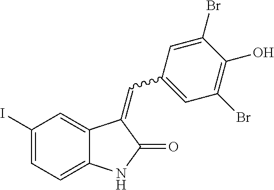 [SMILES: O=C1Nc2ccc(I)cc2C1=Cc1cc(Br)c(O)c(Br)c1]

and isomers or salts thereof.

3-(3,5-Dibromo-4-hydroxy-benzylidene)-5-iodo-1,3-dihydro-indol-2-one is also known as GW5074; 3-(3,5-Dibromo-4-hydroxybenzylidene)-5-iodo-1,3-dihydroindol-2-one; 3-[(3,5-Dibromo-4-hydroxyphenyl)methylene]-1,3-dihydro-5-iodo-2H-indol-2-one; 3-[(3,5-dibromo-4-hydroxyphenyl)methylidene]-5-iodo-1H-indo1-2-one; 8.5-Iodo-3-[(3,5-dibromo-4-hydroxyphenyl)methylene]-2-indolinone; 10.(Z)-3-(3,5-Dibromo-4-hydroxybenzylidene)-5-iodoindolin-2-one; CAS number 220904-83-6 with the molecular formula C₁₅H₈Br₂INO₂ and molecular weight of 520.946 g/mol.

The compounds and compositions of the present invention, may be useful for modulating muscle stem cell function to maintain or increase skeletal muscle function and/or mass in a subject, and/or substantially prevent or reduce muscle wasting in a subject. In particular, to enhance: the number of muscle stem cells, the function of muscle stem cells, myogenesis and muscle growth.

The compounds and compositions of the present invention, may be useful to promote muscle regeneration, recovery from muscle wasting or muscle injury, and/or to prevent or treat sarcopenia or cachexia; or precachexia. In particular, wherein sarcopenia is loss of muscle mass and/or strength linked to aging and cachexia is associated with a disease, for example, when associated with cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease (Von Haehling et al. 2014).

### DESCRIPTION OF THE DRAWINGS

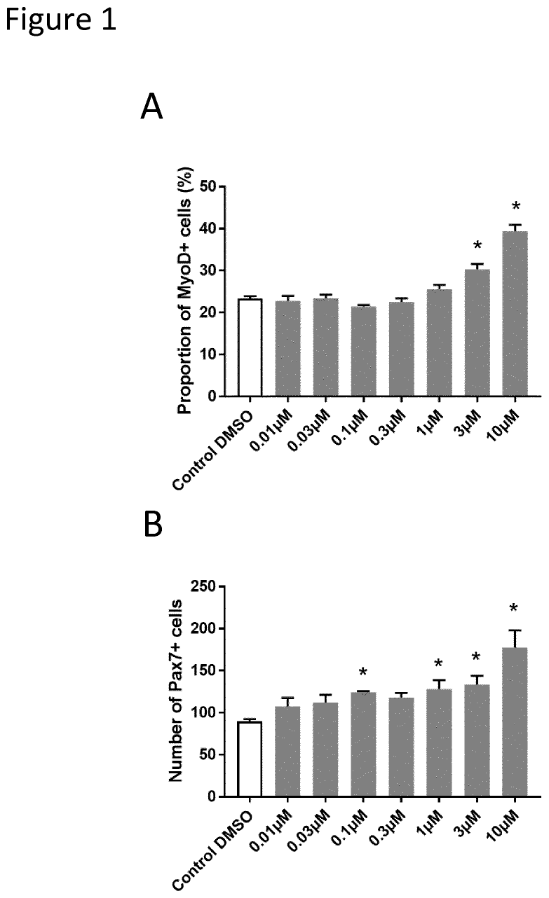

### FIG. 1—Proliferation and Myogenic Commitment of Muscle Stem Cells with GW5074

FIG. 1 demonstrates that GW5074 promoted the expansion of human primary muscle stem cells as shown by the increase in Pax7+ cells, and their myogenic commitment as shown by the increase in the proportion of MyoD+ cells.

FIG. 1A shows the amount of differentiating myoblast (MyoD+) cells as a percent of total cell number in dose-dependent response to compound GW5074. MyoD+ cells represent cells that do not express Pax7 and express only MyoD.

FIG. 1B shows the number of Pax7+ cells in dose dependent response to compound GW5074. Pax7+ cells represent cells that express Pax7 regardless of MyoD expression.

* indicates difference from the control, One-way ANOVA p<0.01, data are presented as Mean+/−SEM

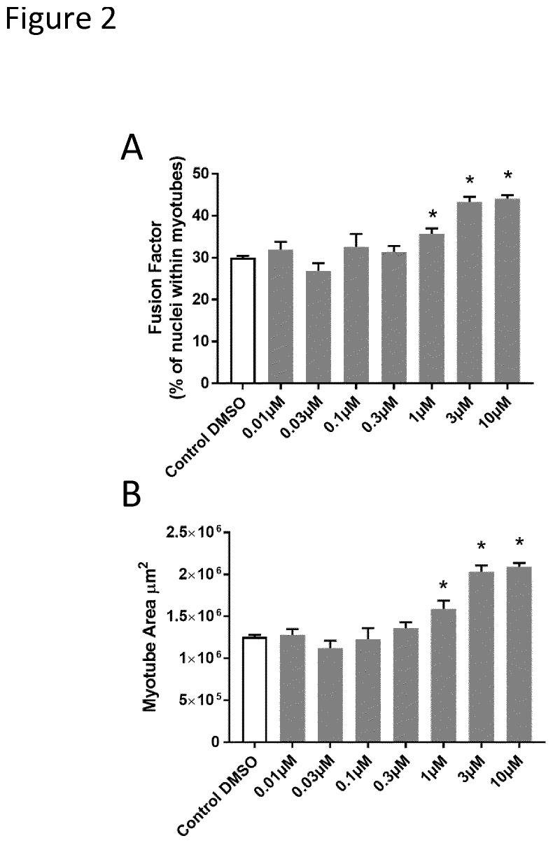

### FIG. 2—Myofiber Growth and Differentiation with GW5074

FIG. 2 shows that GW5074 enhanced myogenic differentiation and myofiber growth of human primary muscle cells.

FIG. 2A shows myogenic differentiation measured by the Fusion Factor as the percent nuclei within troponinT-positive myotubes in a dose dependent response to compound GW5074.

FIG. 2B shows myofiber growth as the area covered by troponinT-positive myotubes in a dose dependent response to compound GW5074.

* indicates difference from the control, One-way ANOVA p<0.01, data are presented as Mean+/−SEM

### DETAILED DESCRIPTION OF THE INVENTION

Various preferred features and embodiments of the present invention will now be described by way of non-limiting examples.

### Compounds of the Invention

Compounds of the present invention are Raf-1 inhibitors. Raf-1 is known also as c-Raf a member of the proto-oncogene serine/threonine protein kinase family.

In one embodiment, the present invention relates to a compound of formula (I):

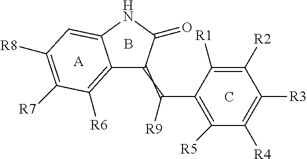

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl.

The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group. The phenyl ring C can be replaced by a pyridyl, pyrimidyl, naphthyl, quinolinyl, or isoquinolinyl group. These groups can be further substituted by H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In another embodiment, the invention relates to compounds of the formula (II):

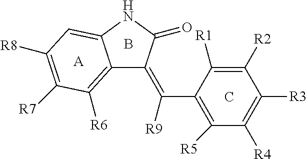

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

The phenyl ring C can be replaced by a pyridyl, pyrimidyl, naphthyl, quinolinyl, or isoquinolinyl group. These groups can be further substituted by H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In another embodiment, the invention relates to compounds of the formula (III):

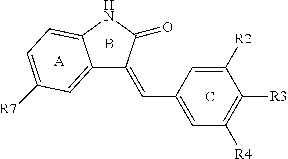

wherein R2, R3, R4, and R7, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid. The alkyl, alkenyl, or alkynyl chain can be substituted by one or two atoms of sulfur included in groups like sulfhydryls, and thioethers. The alkyl, alkenyl, or alkynyl chain can be terminated by a cyanide group.

In a preferred embodiment, compounds of formula (III) wherein R2, R4 and R7 are substituted halogens.

In a preferred embodiment of the invention, the compound is 3-(3,5-Dibromo-4-hydroxy-benzylidene)-5-iodo-1,3-dihydro-indol-2-one:

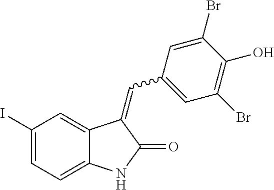 [SMILES: O=C1Nc2ccc(I)cc2C1=Cc1cc(Br)c(O)c(Br)c1]

and isomers or salts thereof.

3-(3,5-Dibromo-4-hydroxy-benzylidene)-5-iodo-1,3-dihydro-indol-2-one is also known as GW5074; 3-(3,5-Dibromo-4-hydroxybenzylidene)-5-iodo-1,3-dihydroindol-2-one; 3-[(3,5-Dibromo-4-hydroxyphenyl)methylene]-1,3-dihydro-5-iodo-2H-indol-2-one; 3-[(3,5-dibromo-4-hydroxyphenyl)methylidene]-5-iodo-1H-indol-2-one; 8.5-Iodo-3-[(3,5-dibromo-4-hydroxyphenyl)methylene]-2-indolinone; 10.(Z)-3-(3,5-Dibromo-4-hydroxybenzylidene)-5-iodoindolin-2-one; CAS number 220904-83-6 with the molecular formula C₁₅H₈Br₂INO₂ and molecular weight of 520.946 g/mol.

In a further embodiment of the invention, isomers of 3-(3,5-Dibromo-4-hydroxy-benzylidene)-5-iodo-1,3-dihydro-indol -2-one and salts thereof are also embodied by the invention.

### General Chemistry Terminology

The term “alkyl” refers to a branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
2) an alkyl chain as defined above that is interrupted by 1-10 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
3) an alkyl chain as defined above that has both 1, 2, 3, 4 or 5 substituents as defined above and is also interrupted by 1-10 atoms (e.g. 1, 2, 3, 4 or 5 atoms) as defined above.

The term “alkenyl” refers to a type of alkyl chain in which two atoms of the alkyl group form a double bond that is not part of an aromatic group. That is, an alkenyl chain contains the pattern R—C(R)═C(R)—R, wherein R refers to the remaining portions of the alkenyl group, which may be the same or different. Non-limiting examples of an alkenyl chain include —CH═CH2, —C(CH3)═CH2, —CH═CHCH3, —C(CH3)═CHCH3, —CH2-CH═C(CH3)2, and —C(CH3)2-CH═CH2. The alkenyl moiety may be branched, straight chain, or cyclic (in which case, it would also be known as a “cycloalkenyl” group).

The term “substituted alkenyl” refers to:

1) an alkenyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
2) an alkenyl chain as defined above that is interrupted by 1-10 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
3) an alkenyl chain as defined above that has both 1, 2, 3, 4 or 5 substituents as defined above and is also interrupted by 1-10 atoms (e.g. 1, 2, 3, 4 or 5 atoms) as defined above.

The term “alkynyl” refers to a type of alkyl chain in which two atoms of the alkyl group form a triple bond. That is, an alkynyl group contains the pattern R—C≡C—R, wherein R refers to the remaining portions of the alkynyl group, which may be the same or different. Non-limiting examples of an alkynyl group include —C≡CH, —C≡CCH3 and —C≡CCH2CH3. The “R” portion of the alkynyl moiety may be branched, straight chain, or cyclic.

The term “substituted alkynyl” refers to:

1) an alkynyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
2) an alkynyl chain as defined above that is interrupted by 1-10 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
3) an alkynyl chain as defined above that has both 1, 2, 3, 4 or 5 substituents as defined above and is also interrupted by 1-10 atoms (e.g. 1, 2, 3, 4 or 5 atoms) as defined above.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system. As used herein, the term “ring system” refers to two or more rings, wherein two or more of the rings are fused. The term “fused” refers to structures in which two or more rings share one or more bonds.

The term “halogen atom” may refer to a fluorine atom, a chlorine atom, a bromine atom or an iodine atom.

The term “naphthalene” refers to a structure consisting of a fused pair of benzene rings.

The term “analogue” as used herein is understood to refer to a compound having a structure similar to that of another one, but differing from it in respect of a certain component. A “derivative” is a compound that can be imagined to arises or is actually be synthesized from a parent compound by replacement of one or more atoms with another atom or group of atoms.

The term “isomer” as used herein is understood to refer to a compound with the same molecular formula but a different arrangement of atoms in the molecule.

Salts are especially the pharmaceutically acceptable salts of compounds of formulae I, II or Ill.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

In one embodiment of the invention, a compound of formula (I) or isomers or salts thereof are provided to maintain or increase skeletal muscle function and/or mass in a subject, and/or substantially prevent or reduce muscle wasting in a subject is represented by formula (I):

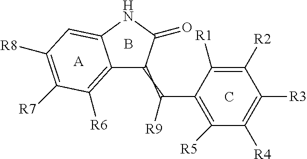

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl.

In another embodiment of the invention, a compound of formula (II) or isomers or salts thereof are provided to maintain or increase skeletal muscle function and/or mass in a subject, and/or substantially prevent or reduce muscle wasting in a subject is represented by formula (II):

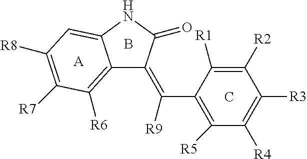

wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl. The alkyl, alkenyl, or alkynyl chain can be substituted by one or two atoms of oxygen included in groups like ethers, primary, secondary and tertiary alcohols, aldehyde, and carboxylic acid

In another embodiment of the invention, a compound or isomers or salts thereof are provided to maintain or increase skeletal muscle function and/or mass in a subject, and/or substantially prevent or reduce muscle wasting in a subject represented by formula (III):

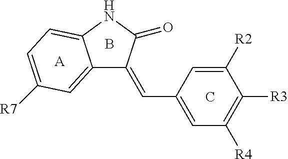

wherein R2, R3, R4, and R7, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, optionally substituted and/or optionally branched, C2 to C10 alkenyl; a linear, optionally substituted and/or optionally branched C2 to C10 alkynyl.

In a preferred embodiment of the invention, the compound provided to maintain or increase skeletal muscle function and/or mass in a subject, and/or substantially prevent or reduce muscle wasting in a subject is 3-(3,5-Dibromo-4-hydroxy-benzylidene)-5-iodo-1,3-dihydro-indol-2-one

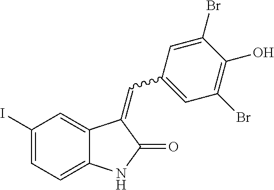 [SMILES: O=C1Nc2ccc(I)cc2C1=Cc1cc(Br)c(O)c(Br)c1]

or isomers or salts thereof.

In one embodiment of the invention, compounds of the invention modulate muscle stem cell function to maintain or increase skeletal muscle function and/or mass in a subject, and/or substantially prevent or reduce muscle wasting in a subject, and/or to enhance muscle repair after an injury, for example by accelerating the repair of myofibers or decreasing fibrosis and muscle stiffness or decreasing muscle fat infiltration.

In another embodiment of the invention, compounds of the invention modulate muscle stem cell function by proliferation and/or differentiation of skeletal muscle stem cells.

In a further embodiment of the invention, compounds of the invention modulate muscle stem cell function by myogenesis.

### Pharmaceutical Compositions

Pharmaceutical compositions, contain, for example, from about 10% to about 100%, preferably from about 20% to about 60%, of the active ingredients. Pharmaceutical preparations for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content contained in an individual dose of each dosage form need not in itself constitute an effective amount since the necessary effective amount can be reached by administration of a plurality of dosage units.

In particular, a therapeutically effective amount of a compound of the invention may be administered simultaneously or sequentially and in any order, and for combinations, the components may be administered separately or as a fixed combination. For example, when used in a method of treatment of cachexia associated with chemotherapy for cancer according to the present invention may comprise (i) administration of the combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein.

The individual combination partners of the invention can be administered separately at different times during the course of therapy or concurrently in divided or single combination forms. The invention is embracing all such regimes of simultaneous or alternating treatment and the term “administering” is to be interpreted accordingly.

The effective dosage may vary depending on the particular compound or pharmaceutical composition employed, the mode of administration, the condition being treated, the severity of the condition being treated. Thus, the dosage regimen is selected in accordance with a variety of factors including the route of administration and the renal and hepatic function of the patient.

A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The compound of formula I, II or III can be administered by any route including orally, parenterally, e.g., intraperitoneal, intravenously, intramuscularly, subcutaneously, intratumorally, or rectally, or enterally. Preferably the compound of formula I, II or III is administered orally, preferably at a daily dosage of 1-300 mg/kg body weight or, for most larger primates, a daily dosage of 50-5000, preferably 500-3000 mg. A preferred oral daily dosage is 1-75 mg/kg body weight or, for most larger primates, a daily dosage of 10-2000 mg, administered as a single dose or divided into multiple doses, such as twice daily dosing.

The compound of formula I, II or III, is preferably administered orally to a human in a dosage in the range of about 100 to 2000 mg/day, more preferably 500 to 1500 mg/day, e.g. 1000 mg/day and most preferably 750 mg/day to 1500 mg/day.

In one embodiment of the invention, the composition of the invention is provided for use to maintain or increase muscle function and/or mass in a subject, and/or substantially prevent or reduce muscle wasting in a subject.

In another embodiment of the invention, the composition is a pharmaceutical composition of the invention or a pharmaceutically acceptable salt thereof is provided for use to maintain or increase muscle function and/or mass in a subject, and/or substantially prevent or reduce muscle wasting in a subject.

In another embodiment of the invention, the composition is a pharmaceutical composition comprising a compound of the invention wherein modulation of muscle stem cell function is measured by increase in the number of muscle stem cells and/or myoblasts and/or myotubes.

In one embodiment of the invention, the pharmaceutical composition or a pharmaceutically acceptable salt thereof is provided to maintain or increase muscle function and/or mass in a subject, and/or substantially prevent or reduce muscle wasting in a subject.

In another embodiment of the invention, the pharmaceutical composition or pharmaceutically acceptable salt thereof is provided to prevent or treat cachexia or precachexia; sarcopenia, myopathy, dystrophy, and/or recovery after muscle injury or surgery.

In a further embodiment of the invention, the pharmaceutical composition of the invention is provided to be used to prevent or treat cachexia wherein cachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In a preferred embodiment of the invention, the pharmaceutical composition of the invention is provided to be used to prevent or treat cachexia or precachexia associated with cancer.

In another preferred embodiment of the invention, the pharmaceutical composition of the invention is provided to be used in the treatment of cachexia associated with cancer is selected from pancreas cancer, esophagus, stomach, bowel, lung and/or liver cancer.

In a further embodiment of the invention, the compound or composition of the invention is provided to be be used in the manufacture of a medicament for the prevention and/or treatment of cachexia.

### Combination Together with Chemotherapy Agents to Treat Cancer

A combination of the invention includes at least one compound of the invention and a chemotherapy agent to treat cancer wherein the active ingredients are present in each case in free form or in the form of a pharmaceutically acceptable salt, and optionally at least one pharmaceutically acceptable carrier.

Administration of a combination results in a surprising beneficial effect of slowing down, arresting or reversing the progress of muscle wasting, e.g. less cachexia, an improved quality of life and a decreased mortality and morbidity, compared to a monotherapy applying only one of the pharmaceutically active ingredients.

In one embodiment of the invention, the pharmaceutical composition of the invention or pharmaceutically acceptable salt thereof may be administered in combination with a therapeutic anti-cancer compound.

### Kit of Parts

A combination preparation can be defined as a “kit of parts” in the sense that it can be dosed independently or by use of different fixed combinations with distinguished amounts of combination i.e. simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners.

In one embodiment of the invention, a kit of parts is provided for the prevention or treatment of cachexia or precachexia comprising a compound or composition of the invention or a pharmaceutically acceptable salt thereof.

In another embodiment of the invention, a kit of parts is provided for the prevention or treatment of cachexia or precachexia comprising a compound of the invention or a pharmaceutically acceptable salt thereof to be administered separately or together with an anti-cancer treatment.

In another embodiment of the invention, a kit of parts is provided for maintaining or increasing muscle function and/or muscle mass in a subject and/or substantially preventing or reducing muscle wasting in a subject with sarcopenia, myopathy, dystrophy and/or recover after muscle injury or surgery comprising a compound or a composition of the invention or a pharmaceutically acceptable salt thereof.

In a further embodiment of the invention, a kit of parts is provided wherein the kit additionally comprises instructions for dietary intervention of high caloric, high protein, high carbohydrate, Vitamin B3, B12 and/or Vitamin D supplementation, antioxidants, omega fatty acids and/or polyphenols for daily administration.

### Combination of Compounds and Compositions of the Invention with Dietary Intervention

The term “dietary intervention” refers to an external factor applied to a subject which causes a change in the subject's diet. In one embodiment, the dietary intervention is a high calorie diet. In another embodiment, the dietary intervention is a high protein and/or carbohydrate diet. In another embodiment, the dietary intervention is a diet supplemented with vitamins and minerals, in particular vitamin B12 and/or vitamin D. In another embodiment the dietary intervention is supplemented with anti-oxidants, for example N-acetyl-cysteine. In a further embodiment, the dietary intervention is supplemented with omega fatty acids. In a further embodiment, the dietary intervention is supplemented with a polyphenol or a vitamin B3 that increases mitochondrial activity, for example nicotinamide riboside.

The diet may be one which is adjusted to the starting body weight of the subject.

The dietary intervention may comprise administration of at least one diet product. The diet product may be a meal replacement product or a supplement product which may, for example, increase the subject's appetite. The diet product may include food products, drinks, pet food products, food supplements, nutraceuticals, food additives or nutritional formulae. Example oral nutritional supplements include Nestlé Boost, Resource and Meritene products.

In one embodiment of the invention, a compound or a composition of the invention may be used in a method of prevention or treatment of cachexia in combination with a dietary intervention of high caloric, high protein, high carbohydrate, Vitamin B3, Vitamin B12 and/or Vitamin D supplementation, antioxidants, omega fatty acids and/or polyphenols.

### Cachexia and Related Diseases

The invention provides compounds, compositions and methods of preventing and/or treating cachexia or skeletal muscle wasting syndrome by modulating skeletal muscle stem cells. Cachexia is a complex metabolic syndrome associated with underlying illness and characterized by loss of muscle with or without loss of fat mass. The prominent clinical feature of cachexia is weight loss in adults (corrected for fluid retention) or growth failure in children (excluding endocrine disorders).

Cachexia is often seen in patients with diseases such as cancer, chronic heart failure, renal failure, chronic obstructive pulmonary disease, AIDS, autoimmune disorders, chronic inflammatory disorders, cirrhosis of the liver, anorexia, chronic pancreatitis and/or metabolic acidosis and neurodegenerative disease.

There are certain types of cancer wherein cachexia is particularly prevalent, for example, pancreas, esophagus, stomach, bowel, lung and/or liver cancer.

The internationally recognised diagnostic criterion for cachexia is weight loss greater than 5%, over a restricted time, for example, 6 months, or weight loss greater than 2% in individuals already showing depletion according to current bodyweight and height (body-mass index [BMI] <20 kg/m²) or skeletal muscle mass (measured by DXA, MRI, CT or bioimpedance). Cachexia can develop progressively through various stages—precachexia to cachexia to refractory cachexia. Severity can be classified according to degree of depletion of energy stores and body protein (BMI) in combination with degree of ongoing weight loss.

In particular, cancer cachexia has been defined as weight loss >5% over past 6 months (in absence of simple starvation); or BMI <20 and any degree of weight loss >2%; or appendicular skeletal muscle index consistent with sarcopenia (males <7.26 kg/m²; females <5.45 kg/m²) and any degree of weight loss >2% (Fearon et al. 2011).

Precachexia may be defined as weight loss ≤5%, together with anorexia and metabolic change. At present there are no robust biomarkers to identify those precachectic patients who are likely to progress further or the rate at which they will do so. Refractory cachexia is defined essentially on the basis of the patient's clinical characteristics and circumstances.

It may be appreciated that the compounds, compositions and methods of the present invention may be beneficial for the prevention and/or treatment of the condition of precachexia as well as cachexia in particular to maintain or improve skeletal muscle mass and/or muscle function.

In one embodiment of the invention, the invention provides a method of treatment of cachexia or precachexia comprising administering to a human or animal subject an effective amount of a compound of the invention or an isomer, or a pharmaceutically acceptable salt thereof.

In another embodiment of the invention, the invention provides a method of treatment of cachexia or precachexia comprising administering to a human or animal subject an effective amount of a compound of the invention or an isomer, or a pharmaceutically acceptable salt thereof wherein cachexia or precachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In a preferred embodiment of the invention, the invention provides a method of treatment of cancer cachexia is associated with cancer is selected from pancreas, esophagus, stomach, bowel, lung and/or liver cancer.

In yet another embodiment of the invention, the invention provides a method of treatment wherein treatment of cancer cachexia is measured by reducing body weight loss, preventing body weight loss, maintaining body weight or increasing body weight.

In another embodiment of the invention, a compound or a composition of the invention may be used in a method of treatment wherein cancer cachexia is a result of treatment for cancer with a chemotherapeutic agent.

In a further embodiment of the invention, a compound or a composition of the invention may be used in a method of prevention or treatment of cachexia in combination with a dietary intervention of high caloric, high protein, high carbohydrate, Vitamin B3, Vitamin B12 and/or Vitamin D supplementation, antioxidants, omega fatty acids, and/or polyphenols.

### Sarcopenia and Related Diseases

Sarcopenia can be characterized by one or more of low muscle mass, low muscle strength and low physical performance.

Sarcopenia can be diagnosed in a subject based on the definition of the AWGSOP (Asian Working Group for Sarcopenia in Older People), for example as described in Chen et al., 2014. Low muscle mass can generally be based on low appendicular lean mass normalized to height square (ALM index), particularly ALM index less than 7.00 kg/m2 for men and 5.40 kg/m2 for women. Low physical performance can generally be based on gait speed, particularly gait speed of <0.8 m/sec. Low muscle strength can generally be based on low hand grip strength, particularly hand grip strength less than 26 kg in men and less than 18 kg in women.

Sarcopenia can be diagnosed in a subject based on the definition of the EWGSOP (European Working Group for Sarcopenia in Older People), for example as described in Crutz-Jentoft et al., 2010. Low muscle mass can generally be based on low appendicular lean mass normalized to height square (ALM index), particularly ALM index less than 7.23 kg/m2 for men and 5.67 kg/m2 for women. Low physical performance can generally be based on gait speed, particularly gait speed of <0.8 m/sec. Low muscle strength can generally be based on low hand grip strength, particularly hand grip strength less than 30kg in men and less than 20 kg in women.

Sarcopenia can be diagnosed in a subject based on the definition of the Foundation for the National Institutes of Health (FNIH), for example as described in Studenski et al., 2014. Low muscle mass can generally be based on low appendicular lean mass (ALM) normalized to body mass index (BMI; kg/m2), particularly ALM to BMI less than 0.789 for men and 0.512 for women. Low physical performance can generally be based on gait speed, particularly gait speed of <0.8 m/sec. Low muscle strength can generally be based on low hand grip strength, particularly hand grip strength less than 26 kg in men and less than 16 kg in women. Low muscle strength can also generally be based on low hand grip strength to body mass index, particularly hand grip strength to body mass index less than 1.00 in men and less than 0.56 in women.

The D3-creatine dilution method is another approach to measure muscle mass. This method is becoming more widely accepted as a robust standard and potentially a future alternative to DXA. The D3-creatine dilution method has been described previously in Clark et al. (1985) and Stimpson et al. (2013).

It may be appreciated that the compounds, compositions and methods of the present invention may be beneficial to prevent and/or treat sarcopenia and/or related conditions, in particular, to maintain or improve skeletal muscle mass and/or muscle function.

### Myopathy and Related Conditions

Myopathies are neuromuscular disorders in which the primary symptom is muscle weakness due to dysfunction of muscle fiber. Other symptoms of myopathy can include include muscle cramps, stiffness, and spasm. Myopathies can be inherited (such as the muscular dystrophies) or acquired (such as common muscle cramps).

Myopathies are grouped as follows: (i) congenital myopathies: characterized by developmental delays in motor skills; skeletal and facial abnormalities are occasionally evident at birth (ii) muscular dystrophies: characterized by progressive weakness in voluntary muscles; sometimes evident at birth (iii) mitochondrial myopathies: caused by genetic abnormalities in mitochondria, cellular structures that control energy; include Kearns-Sayre syndrome, MELAS and MERRF glycogen storage diseases of muscle: caused by mutations in genes controlling enzymes that metabolize glycogen and glucose (blood sugar); include Pompe's, Andersen's and Cori's diseases (iv) myoglobinurias: caused by disorders in the metabolism of a fuel (myoglobin) necessary for muscle work; include McArdle, Tarui, and DiMauro diseases (v) dermatomyositis: an inflammatory myopathy of skin and muscle (vi) myositis ossificans: characterized by bone growing in muscle tissue (vii) familial periodic paralysis: characterized by episodes of weakness in the arms and legs (viii) polymyositis, inclusion body myositis, and related myopathies: inflammatory myopathies of skeletal muscle (ix) neuromyotonia: characterized by alternating episodes of twitching and stiffness; and stiff-man syndrome: characterized by episodes of rigidity and reflex spasms common muscle cramps and stiffness, and (x) tetany: characterized by prolonged spasms of the arms and legs. (Reference: https://www.ninds.nih.gov/disorders/all-disorders/myopathy-information-page).

It may be appreciated that the compounds, compositions and methods of the present invention may be beneficial to prevent and/or treat the aforementioned diseases or conditions, in particular, to maintain or improve skeletal muscle mass and/or muscle function.

### Muscular Dystrophy

Muscular dystrophy are a group of genetic diseases characterized by progressive weakness and degeneration of the skeletal or voluntary muscles which control movement. Major types of muscular dystrophy include: Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy and myotonic dystrophy.

(Reference: https://www.medicalnewstoday.com/articles/187618.php)

It may be appreciated that the compounds, compositions and methods of the present invention may be beneficial to prevent and/or treat the aforementioned diseases or conditions, in particular, to maintain or improve skeletal muscle mass and/or muscle function,

### Recovery After Muscle Injury from Surgery and Muscle Traumas

Muscle injuries can be caused by bruising, stretching or laceration causing acute or chronic soft tissue injury that occurs to a muscle, tendon, or both. It may occur as a result of fatigue, overuse, or improper use of a muscle. It may occur after physical trauma such as a fall, fracture or overuse during physical activity. Muscle injuries may also occur after surgery such as joint replacement arthroscopic surgery.

It may be appreciated that the compounds, compositions and methods of the present invention may be beneficial to prevent and/or treat the aforementioned conditions of recovery after surgery and/or muscle trauma, in particular, to maintain or improve skeletal muscle mass and/or muscle function.

### Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder. As used herein, a subject is “in need of a treatment if such subject would benefit biologically, medically or in quality of life from such treatment.

### Subject

The term “subject” means any animal, including humans and companion animals. Generally, the subject is a human or an avian, bovine, canine, equine, feline, hircine, murine, ovine or porcine animal. The subject can be a horse or a companion animal, for example a cat or a dog. Preferably, the subject is a human.

The treatment of mammals, particularly humans, is preferred. However, both human and veterinary treatments are within the scope of the invention.

For veterinary subjects, dogs, cats and equine subjects are preferred.

The present invention may also be useful in non-human animal subjects such as: avian, bovine, ovine or porcine animals, for optimizing meat production by increasing skeletal muscle mass and/or function.

### Muscle Stem Cells

The term “muscle stem cell”, as used herein, may refer to satellite cells, preferably satellite cells that are quiescent and are uncommitted.

Satellite cells are precursors to skeletal muscle cells. In adult muscle, satellite cells are generally quiescent, but can activate and undergo myogenesis in response to disease or mechanical strain such as injury or exercise. Satellite cells are also involved in the normal growth of muscle. Upon activation, satellite cells can proliferate before undergoing myogenic differentiation to finally fuse with existing myofibers or to form new myofibers, depending on the magnitude of tissue trauma. In addition to generating differentiated myogenic progeny, at least some satellite cells can self-renew, thereby meeting the defining criteria of bona fide resident stem cells. Pax7 is the most well-known and characterized marker express by muscle stem cells i.e. muscle stem cells can be reliably identified based on their expression of the paired box transcription factor Pax7. The muscle stem cells may also express NCAM, CD56, CD29 and/or CD82, i.e. the muscle stem cells may be NCAM+, CD56+, CD29+ and/or CD82+.

MyoD+ is a commitment marker that may be used to distinguish quiescent from committed satellite cells.

### Muscle Function and Mass

The compounds, compositions, uses and methods disclosed herein may provide for the maintenance of or increase in muscle function and/or mass.

The term “muscle function” refers to the ability of a muscle to perform in a manner that does not negatively impact on the life of a subject, and encompasses parameters of muscle strength, muscle contraction, muscle endurance and/or muscle elasticity.

Suitable tests for assessing muscle function include grip strength assessment using a dynamometer; one repeat maximum on leg press, chest press or leg extension; gait speed; 6 min walk test; time up and go; short physical performance battery; Fried frailty criteria; and stair climbing time assessments.

Muscle mass (which may equate with muscle volume, muscle thickness or myofiber size) may be measured by dual-energy X-ray absorptiometry (DXA) or bioimpedance tests. Similarly, MRI may be used for assessing muscle volume and ultra-sound may be used for assessing muscle thickness and pennation angle.

“Muscle wasting” may be a reduction in muscle mass, for example to the stage where the muscle loss becomes debilitating. In one embodiment, the subject does not lose more than 10%, 5%, 4%, 3%, 2% or 1% of their muscle mass.

Preferably, the compounds, compositions, uses and methods disclosed herein provide for the maintenance of or increase in muscle mass.

The term “maintains” refers to a particular parameter, such as muscle function and/or mass, remaining substantially unchanged over a period of time (e.g. 5, 10, 15, 20, 25, 30, 40, 50 or more years).

In one embodiment, muscle mass increases by at least 1%, 2%, 3%, 4%, 5%, 10%, 15% or 20%.

In another embodiment, muscle mass increases by 1-2.5%, 1-5%, 1-10% or 1-20%. Preferably, the muscle is skeletal muscle.

### EXAMPLES

### Example 1: Selection of Compounds modulating Muscle Stem Cells

### Selection of Human Skeletal Muscle Myoblasts

The inventors developed a high content screening to test in vitro compounds on human primary adult muscle cells. Human Skeletal Muscle Myoblasts (HSMM) were purchased from Lonza (https://bioscience.lonza.com). These cells were isolated from the upper arm or leg muscle tissue of normal donors and used after the second passage. Several donors have been tested to ensure cell viability and purity before selecting the final donor which was a 36-year-old Caucasian female.

### Assay for Muscle Stem Cell Commitment

The primary screening assay was based on the high content detection of two important myogenic regulatory factors (Pax7 and MyoD) by immunofluorescence. Pax7 and MyoD are the major hallmarks of muscle stem cell stemness and commitment and can be used to monitor muscle stem cell progeny. In particular, Pax7 marks early amplification while MyoD is a later marker for myogenic commitment, and combinations of these markers define the different states of proliferation, differentiation and self-renewal.

The hit selection was primarily based on compounds that can enhance the commitment toward the myogenic differentiation (Pax7−/MyoD+ cells), which is particularly relevant in the context of cancer cachexia where a defect in myogenic commitment has been revealed as a potential cause of the muscle wasting (He et al. 2013).

Human primary myoblasts were seeded in 384 well plates at a density of 1'000 cells per well in skeletal muscle growth medium (SKM-M, AMSbio). For treatment, compounds were directly added to the myoblast cultures 16 hours after initial plating. All cultures were then grown for 96 hours. Cells were stained for Pax7 and MyoD expression using antibodies directed against Pax7 and MyoD and counterstained with Hoechst 33342 to visualize cell nuclei. MyoD+ are defined as cells that do not express Pax7 but express MyoD. Image acquisition was performed using the ImageXpress (Molecular Devices) platform. Custom module analysis based on Multi-Wavelength Cell Scoring of the MetaXpress software was used for quantification. FIG. 1 shows for the number of Pax7+ cells and MyoD+ cells normalized to the total cell number in order to evaluate the myogenic commitment.

### Assay for Myogenesis

In order to confirm the results from the primary assay described above, a secondary assay for myogenesis in vitro on all the hits obtained in this primary screening assay. This secondary assay focused on the later stage of myogenesis when the muscle cells fuse together and form multinucleated myotubes. This assay was based on the detection of the mature troponin-T protein that is expressed in myotubes. In particular, two measurements were made:

Fusion factor, which is the % of nuclei that are inside the multinucleated myotubes compare to total nuclei and give a readout for muscle cell differentiation.

Myotube area, which is measured based on the Troponin T staining and give a readout on the myotube size and muscle fiber growth.

Human primary myoblasts were seeded in 384 well plates at a density of 3'000 cells per well in skeletal muscle growth medium (SKM-M, AMSbio). After one day, the differentiation is induced by a medium change. For treatment, compounds were directly added to the myoblast cultures for 96 hours. Myotubes were stained for TroponinT expression using antibodies directed against TroponinT and counterstained with Hoechst 33342 to visualize cell nuclei. Image acquisition was performed using the ImageXpress (Molecular Devices) platform. Custom module analysis based on Multi-Wavelength Cell Scoring of the MetaXpress software was used for quantification. FIG. 2 shows the viability of the cells assessed by cell number, myogenic differentiation measured by the Fusion Factor as the percent nuclei within troponinT-positive myotubes, and myofiber growth as the area covered by troponinT-positive myotubes.

### Example 2: Clinical Trial with Compound GW5074

Ongoing Phase I/IIa clinical study with GW5074, subjects receive a dosing regimen of the combination therapy of GW5074 and sorafenib (within the safe dosing range defined at Phase I) for ≥168 days (24 weeks). The dosage of GW5074: 750 mg to 1500 mg (daily dose).

https://clinicaltrials.gov/ct2/show/NCT03406364

### REFERENCES

Almeida et al. (2016) Muscle Satellite Cells: Exploring the Basic Biology to Rule Them, Stem Cells International, Vol. 2016, ID 1078686.
Chen, L. K., et al. (2014). Sarcopenia in Asia: consensus report of the Asian Working Group for Sarcopenia. Journal of the American Medical Directors Association 15, 95-101.
Clark R V, Walker A C, O'Connor-Semmes R L, Leonard M S, Miller R R, Stimpson S A, Turner S M, Ravussin E, Cefalu W T, Hellerstein M K, Evans W J (1985). Total body skeletal muscle mass: estimation by creatine (methyl-d3) dilution in humans. J Appl Physiol. June 15; 116(12):1605-13.
Cruz-Jentoft, A. J., Baeyens, J. P., Bauer, J. M., Boirie, Y., Cederholm, T., Landi, F., Martin, F. C., Michel, J. P., Rolland, Y., Schneider, S. M., et al. (2010). Sarcopenia: European consensus on definition and diagnosis: Report of the European Working Group on Sarcopenia in Older People. Age Ageing 39, 412-423.
Fearon et al. (2011) Definition and classification of cancer cachexia: an international consensus. Lancet Oncology, 12, 489-495.
He W A, Berardi E, Cardillo V M, Acharyya S, Aulino P, Thomas-Ahner J, Wang J, Bloomston M, Muscarella P, Nau P, Shah N, Butchbach M E, Ladner K, Adamo S, Rudnicki M A, Keller C, Coletti D, Montanaro F, Guttridge D C (2013). NF-κB-mediated Pax7 dysregulation in the muscle microenvironment promotes cancer cachexia. J Clin Invest. November; 123(11):4821-35.
Studenski S A, Peters K W, Alley D E, Cawthon P M, McLean R R, Harris T B, Ferrucci L, Guralnik J M, Fragala M S, Kenny A M, Kiel D P, Kritchevsky S B, Shardell M D, Dam T T, Vassileva M T (2014). The FNIH sarcopenia project: rationale, study description, conference recommendations, and final estimates. J Gerontol A Biol Sci Med Sci. 69(5), 547-558.
Stimpson S A, Leonard M S, Clifton L G, Poole J C, Turner S M, Shearer T W, Remlinger K S, Clark R V, Hellerstein M K, Evans W J. (2013) Longitudinal changes in total body creatine pool size and skeletal muscle mass using the D<sub>3</sub>-creatine dilution method. J Cachexia Sarcopenia Muscle. June 25.
von Haehling, S. and S. D. Anker, Prevalence, incidence and clinical impact of cachexia: facts and numbers-update (2014). J Cachexia Sarcopenia Muscle, 5(4): p. 261-3 (2014).

1. A method to maintain or increase skeletal muscle function and/or mass in a subject, and/or substantially prevent or reduce muscle wasting in a subject comprising the step of administering to a subject in need of same a composition comprising compound of formula (I) or isomers or salts thereof represented by formula (I): 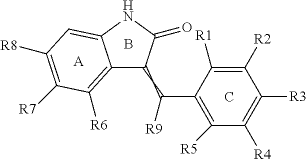
wherein R1, R2, R3, R4, R5, R6, R7, R8, and R9, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, C2 to C10 alkenyl; a linear, branched C2 to C10 alkynyl.

2. A method according to claim 1 to maintain or increase skeletal muscle function and/or mass in a subject, and/or substantially prevent or reduce muscle wasting in a subject comprising the step of administering to a subject in need of same a composition comprising represented by formula (III): 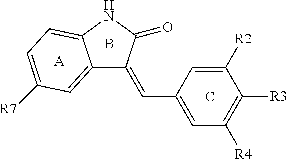
wherein R2, R3, R4, and R7, are each independently H; OH; OMe; O-alkyl; SH; S-Me; S-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, C2 to C10 alkenyl; a linear, C2 to C10 alkynyl.

3. A method according to claim 1 wherein the compound is 3-(3,5-Dibromo-4-hydroxy-benzylidene)-5-iodo-1,3-dihydro-indol-2-one 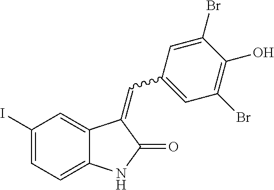 [SMILES: O=C1Nc2ccc(I)cc2C1=Cc1cc(Br)c(O)c(Br)c1]
or isomers or salts thereof.

4. A method according claim 1 to maintain or increase muscle function and/or mass in a subject and/or substantially prevent or reduce muscle wasting in a subject by modulating muscle stem cell function.

5. A method according to claim 1 to maintain or increase muscle function and/or mass in a subject, and/or substantially prevent or reduce muscle wasting in a subject.

6. (canceled)

7. A method according to claim 1 to prevent or treat cachexia or precachexia; sarcopenia, myopathy, dystrophy, and/or recovery after muscle injury or surgery.

8. A method according to claim 7 wherein cachexia is associated with a disease selected from the group consisting of cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and neurodegenerative disease.

9-19. (canceled)

20. A kit for the prevention or treatment of cachexia or precachexia comprising a compound or a pharmaceutically acceptable salt thereof a method to maintain or increase skeletal muscle function and/or mass in a subject, and/or substantially prevent or reduce muscle wasting in a subject comprising the step of administering to a subject in need of same a composition comprising compound of formula (I) or isomers or salts thereof represented by formula (I): 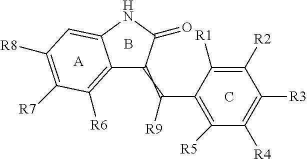
wherein R1, R2, R3, R4, R5, R6, R7, R8, and R9, are each independently H; OH; OMe; O-alkyl; SH; S-Me; 5-alkyl; a halogen; a primary, secondary, or tertiary alcohol, a ketone, an aldehyde; a carboxylic acid; a primary, secondary, or tertiary amine; a primary or secondary amide; a cyano; an alkyl cyanide; a nitro; a sulfonate; a sulfate; a linear, C2 to C10 alkenyl; a linear, branched C2 to C10 alkynyl to be administered separately or together with an anti-cancer treatment.

21. (canceled)

22. A kit according to claim 20 wherein the kit comprises additionally dietary intervention of high caloric, high protein, high carbohydrate, Vitamin B3, B12 and/or Vitamin D supplementation, antioxidants, omega fatty acids and/or polyphenols and instructions for daily administration.
